(19) United States
(12) Patent Application Publication
(10) Pub. No.: US 2005/0165257 A1
(43) Pub. Date: Jul. 28, 2005

(54) Process for preparation of voglibose

(75) Inventors: Takeshi Shogaki, Osaka-shi (JP); Takao Kakita, Osaka-shi (JP); Suguru Yagi, Osaka-shi (JP)

Correspondence Address: BIRCH STEWART KOLASCH & BIRCH, PO BOX 747, FALLS CHURCH, VA, 22040-0747, US

(73) Assignee: SAWAI PHARMACEUTICAL CO., LTD., Osaka-shi (JP)

(21) Appl. No.: 10/509,059

(22) PCT Filed: Oct. 15, 2002
(86) PCT No.: PCT/JP02/10687

(30) Foreign Application Priority Data
Mar. 27, 2002 (JP) 2002-088321

(51) Int. Cl. C07C 213/00

(52) U.S. Cl. 564/462

(57) ABSTRACT

A process capable of conveniently preparing voglibose at a low cost in a safe process, and an intermediate which can be suitably used in the process and a process for preparing the intermediate are provided. An inositol derivative represented by the formula (VI): 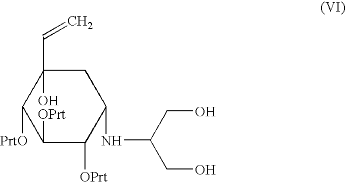
wherein Prt is a protecting group of hydroxyl group; a process for preparing the inositol derivative, wherein a cyclohexanone compound represented by the formula (IV): 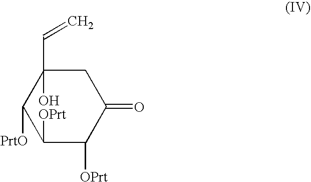 wherein Prt is as defined above,
is dihydroxyaminated using a dihydroxyaminating agent and a reducing agent; and a process for preparing voglibose represented by the formula (VIII): 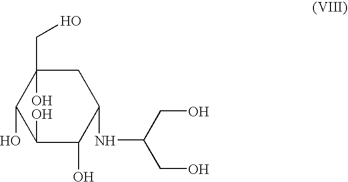 wherein the inositol derivative is oxidized to give an inositol compound, and the protecting group, Prt of the inositol compound is deprotected.

### TECHNICAL FIELD

This invention relates to a novel inositol derivative and a process for their preparation, and a process for preparing voglibose using said inositol derivative as the intermediate. Voglibose is useful as an α-glucosidase inhibitor for the treatment of diabetes.

### BACKGROUND ART

A process for preparing voglibose comprising the following steps have been known:
1) converting validamycin to valienamine by adding validamycin to a culture medium of microorganisms (JP Hei 2-2589 B2),
2) preparing valiolamine using said valienamine (JP Hei 3-16334 B2), and
3) preparing voglibose using said valiolamine (JP Hei 2-38580 B2).

However, above process has serious problems in the costs, product's purity and the safety for human, so that it is not suitable for industrial production. For example, above step 1) requires a lot of labor and time for purifying valienamine from a large amount of the culture supernatant. As for step 2), it is very difficult to exclude the valienamine from valiolamine because of their similar hydrophilicity. In addition, step 3) requires sodium cyanoborohydride (NaBH₃CN) which is a highly toxic substance.

A chemical synthetic process of voglibose from glucose has also been known (JP 2593677 B).

However, it solves above mentioned problems only partially, so that it still contains problems in safety for human and the environment, i.e. it requires special equipments for handling highly toxic trifluoroacetic acid and tri-n-butyl tin, which are used as dehalogenating agents, to ensure the safety for human and to control the liquid waste from the manufacturing plant.

### DISCLOSURE OF INVENTION

This invention is based upon above mentioned prior arts. Its objectives of the present invention are to provide a process for preparing voglibose safely at low costs, and to provide a suitable intermediate for said process and its manufacturing process.

Specifically, this invention relates to:
(1) an inositol derivative represented by the formula (VI): 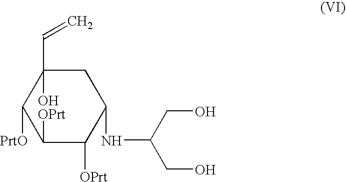
   wherein Prt is a protecting group of hydroxyl group;
(2) a process for preparing an inositol derivative represented by the formula (VI): 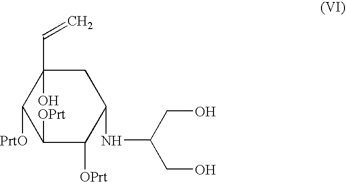
   wherein Prt is a protecting group of hydroxyl group,
   characterized in that a cyclohexanone compound represented by the formula (IV): 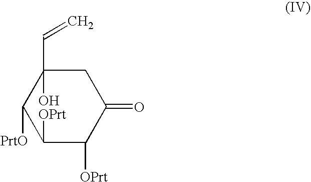
   wherein Prt is as defined above,
   is dihydroxyaminated using a dihydroxyaminating agent and a reducing agent; and
(3) a process for preparing voglibose represented by the formula (VIII): 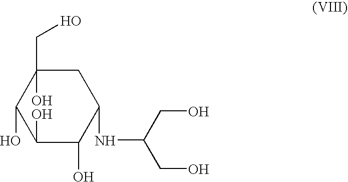
   characterized in that an inositol derivative represented by the formula (VI): 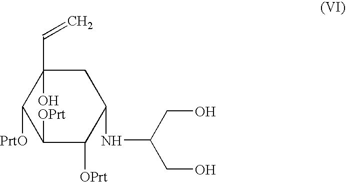
   wherein Prt is a protecting group of hydroxyl group,
   is oxidized to give an inositol compound represented by the formula (VII): 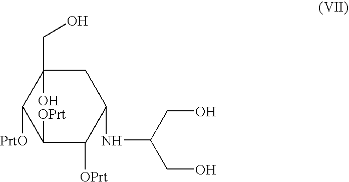
   wherein Prt is as defined above, and
   the protecting group, Prt of the inositol compound is deprotected.

### BEST MODE FOR CARRYING OUT THE INVENTION

The inositol derivative represented by the formula (VI) of the present invention can be prepared by using a hexenopyranoside derivative represented by the formula (I): 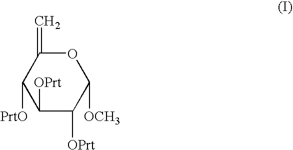
wherein Prt is as defined above,
as a starting material.

The hexenopyranoside derivative represented by the formula (I) is inexpensive and easily available. For instance, the hexenopyranoside derivative can be easily prepared from glucose in accordance with the process described in J. Org. Chem., 1994, 59, 3135-3141.

In the formula (I), Prt is a protecting group of hydroxyl group. Representative examples of Prt include a benzyl group, an acyl group, a silyl group or the like, each of which may have a substituent. The substituent includes, for instance, alkyl groups having 1 to 4 carbon atoms such as methyl group, ethyl group, isopropyl group and tert-butyl group; alkoxy groups having 1 to 4 carbon atoms such as methoxy group; nitro group; and the like, without intending to limit the present invention to those exemplified above.

Specific examples of Prt include benzyl group, benzoyl group, tert-butyldimethylsilyl group, triethylsilyl group, acetyl group, p-methoxybenzyl group, o-nitrobenzyl group and the like, without intending to limit the present invention to those exemplified above. Among them, benzyl group and p-methoxybenzyl group are preferable.

Representative examples of the hexenopyranoside derivative represented by the formula (I) include methyl 6-deoxy-2,3,4-tris-O-(phenylmethyl)-α-D-xylo-5-hexenopyranoside, methyl 6-deoxy-2,3,4-tris-O-[(4-methoxyphenyl)methyl]-α-D-xylo-5-hexenopyranoside, and the like.

First, a cyclohexanone derivative represented by the formula (II): 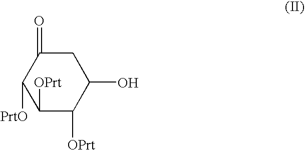
wherein Prt is as defined above,
is prepared by using the hexenopyranoside derivative represented by the formula (I). The process for preparing a cyclohexanone derivative represented by the formula (II) from the hexenopyranoside derivative represented by the formula (I) includes, for instance, a process described in Tetrahedron Letters, 1996, 37, 649-652, and the like.

More specifically, the cyclohexanone derivative represented by the formula (II) can be prepared by, for instance, carrying out Ferrier rearrangement of the hexenopyranoside derivative represented by the formula (I) in a solvent in the presence of a catalyst.

The catalyst includes, for instance, mercury compounds, palladium compounds, nickel compounds and the like. Among them, the palladium compounds are preferable, and palladium chloride is more preferable.

As the solvent, there can be used, for instance, water, tetrahydrofuran, dioxane, acetone or the like. Among them, a mixed solvent of water and dioxane and a mixed solvent of water and acetone are preferable.

It is preferable that the Ferrier rearrangement of the hexenopyranoside derivative represented by the formula (I) is carried out at 20° to 100° C., especially 40° to 60° C.

Next, an inositol derivative represented by the formula (III): 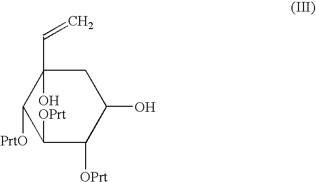
wherein Prt is as defined above,
is prepared from the cyclohexanone derivative represented by the formula (II) in accordance with a process described in, for instance, Carbohydrate Research, 1990, 205, 283-291. More specifically, the inositol derivative represented by the formula (III) can be obtained by carrying out the addition reaction of the cyclohexanone derivative represented by the formula (II).

The addition reaction of the cyclohexanone derivative represented by the formula (II) can be carried out in an appropriate solvent in the presence of an alkenylating agent.

The alkenylating agent includes, for instance, vinyl magnesium bromide and the like.

The solvent includes, for instance, tetrahydrofuran, 1,2-diethoxyethane, diethoxymethane, hexane, toluene and the like. These can be used alone or in admixture of two or more kinds, respectively. Among them, toluene is preferable.

When the addition reaction of the cyclohexanone derivative represented by the formula (II) is carried out, it is preferable that the reaction temperature is −78° to 100° C., especially −78° C. to room temperature.

It is preferable that all reactions are carried out under an inert gas such as nitrogen gas or argon gas.

Next, a cyclohexanone compound represented by the formula (IV): 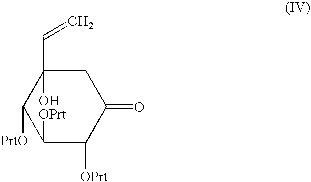
wherein Prt is as defined above,
can be obtained from the resulting inositol derivative represented by the formula (III) in accordance with a process described in, for instance, Carbohydrate Research, 1990, 205, 283-291. More specifically, the cyclohexanone compound represented by the formula (IV) can be obtained by oxidizing the inositol derivative represented by the formula (III) in an appropriate solvent.

The solvent includes, for instance, dimethylformamide, dimethyl sulfoxide, methylene chloride, tetrahydrofuran and the like. Among them, dimethyl sulfoxide is preferable.

The oxidizing agent includes, for instance, sulfur trioxide-pyridine complex, dimethyl sulfoxide-oxalyl chloride, dimethyl sulfoxide-acetic anhydride, dimethyl sulfoxide-trifluoroacetic acid anhydride, dimethyl sulfoxide, dicyclohexyl carbodiimide, pyridinium chlorochromate (PCC), pyridinium dichromate (PDC), tetra-n-propylammonium perruthenate (TPAP), 2,2,6,6-tetramethyl-1-piperidinyloxy(TEMPO)-sodium perchlorate, and the like. Among them, sulfur trioxide-pyridine complex is preferable.

It is preferable that the inositol derivative represented by the formula (III) is reacted with the oxidizing agent at usually −78° to 40° C., especially 0° to 40° C.

Next, the inositol derivative represented by the formula (VI) of the present invention can be obtained by dihydroxyaminating the resulting cyclohexanone compound represented by the formula (IV).

The dihydroxyamination of the cyclohexanone compound represented by the formula (IV) can be carried out by using a dihydroxyaminating agent and a reducing agent in a solvent.

The dihydroxyaminating agent includes, for instance, 2-amino-1,3-propanediol represented by the formula (V): 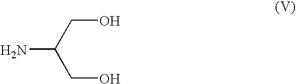
and its derivatives. Preferred examples of the derivatives of 2-amino-1,3-propanediol include, for instance, 2,2-dimethyl-1,3-dioxane-5-amine and the like. Among them, 2-amino-1,3-propanediol is preferable.

It is desirable that the amount of the dihydroxyaminating agent is 1 to 5 mol, preferably 2 to 3 mol per one mol of the cyclohexanone compound represented by the formula (IV).

The solvent includes, for instance, methanol, ethanol, hexane, toluene, ethyl acetate, dichloromethane, chloroform and the like, without intending to limit the present invention to those exemplified above. Among these solvents, methanol is preferable. The amount of the solvent is not limited to specified ones. It is preferable that the amount of the solvent is usually the same volume as the cyclohexanone compound represented by the formula (IV) to 20 volumes.

The reducing agent includes, for instance, borane derivatives such as sodium borohydride and borane; lithium aluminum hydride; palladium catalysts such as palladium carbon and palladium hydroxide; and the like. Among them, sodium borohydride is preferable.

It is desirable that the amount of the reducing agent is 1 to 10 mol, preferably 3 to 5 mol per one mol.

The dihydroxyamination of the cyclohexanone compound represented by the formula (IV) can be carried out by, for instance, dissolving the cyclohexanone compound represented by the formula (IV) and a dihydroxyaminating agent in a solvent, and thereafter adding a reducing agent to the resulting solution. In this case, it is desirable that the temperature of the reaction mixture is −10° to 30° C., preferably −10° C. to room temperature.

The dihydroxyamination can be carried out by maintaining the temperature of the reaction mixture obtained by adding the reducing agent to the above-mentioned solution at 0° to 50° C., preferably 0° to 30° C. with stirring when necessary. The reaction time is not limited to specified ones. The reaction time is usually about 1 to about 24 hours.

Thus, the inositol derivative represented by the formula (VI) can be obtained. The inositol derivative can be isolated by evaporating the solvent from the resulting reaction mixture, adding water to the residue, and extracting it by ethyl acetate or the like.

The resulting inositol derivative represented by the formula (VI) is a white solid, and can be suitably used as an intermediate of voglibose.

Representative examples of the inositol derivative represented by the formula (IV) include 3,4-dideoxy-2-C-ethenyl-4-[[2-hydroxy-1-(hydroxymethyl)ethyl]amino]-1,5,6-tris-O-(phenylmethyl)-D-epi-inositol and the like.

Next, the voglibose represented by the formula (VIII) can be obtained by using the inositol derivative represented by the formula (VI) as an intermediate.

More specifically, the voglibose represented by the formula (VIII) can be obtained by, for instance, the following process.

First, the inositol derivative represented by the formula (VI) is oxidized. The oxidation of the inositol derivative can be carried out by, for instance, dissolving the inositol derivative in a solvent, and blowing ozone into the resulting solution for ozone oxidation.

The solvent includes, for instance, methanol, ethanol, water, ethyl acetate, dimethylformamide, hexane, methylene chloride and the like. Among them, a combined use of methanol and methylene chloride is preferable.

The concentration of the inositol derivative in the solution is not limited to specified ones. It is preferable that the concentration is usually 5 to 30 w/v %.

The end point of the ozone oxidation can be confirmed by, for instance, the disappearance of spots of the inositol derivative by thin-layer chromatography.

After the termination of the oxidation of the inositol derivate, in order to decompose ozonide, it is preferable that a reducing agent, for instance, a borane derivative such as sodium borohydride or borane, or lithium aluminum hydride is added to the above-mentioned solution. In this case, it is desirable that the amount of the reducing agent is 3 to 10 mol, preferably 4 to 6 mol per one mol of the inositol derivative represented by the formula (VI).

Next, it is preferable that the pH of the resulting solution is adjusted to 3 to 5 in order to decompose excess reducing agent. pH adjusting agent includes, for instance, diluted hydrochloric acid, phosphoric acid, acetic acid and the like, without intending to limit the present invention only to those exemplified above.

After that, it is desirable that the pH of this solution is adjusted to 8 to 12, preferably 10 to 12 with an aqueous alkali such as an aqueous sodium hydroxide in order to extract the resulting inositol compound represented by the formula (VII).

Thus, the inositol compound represented by the formula (VII) can be obtained by, for instance, washing with an aqueous sodium chloride or the like, extracting with chloroform or the like, and if necessary, purifying it. This inositol compound can be obtained as a white solid compound.

Representative examples of this inositol compound include 3,4-dideoxy-4-[[2-hydroxy-1-(hydroxymethyl)ethyl]amino]-2-C-(hydroxymethyl)-1,5,6-tris-O-(phenylmethyl)-D-epi-inositol and the like.

Next, the voglibose represented by the formula (VIII) can be obtained by deprotecting a protecting group Prt of the resulting inositol compound with a catalyst.

The deprotection of the protecting group (Prt group) of the inositol compound can be carried out by dissolving the inositol compound in a solvent, and adding a catalyst and a hydrogen source thereto.

As the solvent, there can be used, for instance, an alcohol such as ethanol. The amount of the solvent is not limited to specified ones. The amount may be usually about 5 to about 30 mL per one gram of the inositol compound.

As the catalyst, there can be used, for instance, palladium-carbon, palladium-black, platinum oxide, Raney nickel and the like. Among them, palladium-black is preferable. The amount of the catalyst is not limited to specified ones. The amount can be usually about 100 to about 1000 mg per one gram of the inositol compound.

As the hydrogen source, there can be used, for instance, a hydrogenating agent such as formic acid or ammonium formate, or hydrogen gas under pressure.

The amount of the hydrogen source is not limited to specified ones, and the amount may be one that is usually used. The atmosphere in which the deprotection of the protecting group is carried out is not limited to specified ones. For instance, an inert gas such as argon gas or nitrogen gas is preferable.

In addition, it is desirable that the temperature of the reaction solution is 0° to 100° C., preferably room temperature to 60° C. Thus, the voglibose represented by the (VIII) can be obtained by deprotecting a protecting group Prt of the inositol compound.

The resulting voglibose can be isolated and collected, for instance, by usual procedures such as filtration, concentration, washing, extraction and purification.

The voglibose thus obtained can be suitably used as an α-glucosidase inhibitor in the treatment of diabetes.

According to the present invention, the inositol derivative, which is an intermediate of voglibose, can be obtained selectively in a high yield using an inexpensive and easily available hexenopyranoside derivative as a starting material. Further, the protecting group Prt of the inositol derivative is deprotected to obtain voglibose. The resulting voglibose is easy to purify using the difference of hydrophilicity between the inositol derivative and voglibose.

Next, the present invention will be described more specifically on the bases of Examples, without intending to limit the present invention only to the Examples.

### PREPARATION EXAMPLE 1

### Preparation of Mixture of [2S-(2α,3β,4α,5α)]-5-hydroxy-2,3,4-tris(phenylmethoxy)-cyclohexanone and [2S-(2α,3β,4α,5β)]-5-hydroxy-2,3,4-tris(phenylmethoxy)-cyclohexanone

39 g of palladium chloride was added to a suspension of 2.0 g of methyl 6-deoxy-2,3,4-tris-O-(phenylmethyl)-α-D-xylo-5-hexenopyranoside in 60 mL of dioxane and 30 mL of water at room temperature, and the mixture was stirred at 45° C. for 16 hours.

After the termination of the reaction, 100 mL of water was added to the reaction mixture, and the mixture was extracted with ethyl acetate (100 mL, once). Next, its organic phase was washed with water (100 mL, once), and the mixture was dried over anhydrous sodium sulfate. The desiccant was removed by filtration, and the solvents were evaporated at reduced pressure. The residue was crystallized from 5 mL of ethyl acetate and 50 mL of n-hexane to give 1.40 g of a mixture of [2S-(2α,3β,4α,5α)]-5-hydroxy-2,3,4-tris(phenylmethoxy)-cyclohexanone and [2S-(2α,3β,4α,5β)]-5-hydroxy-2,3,4-tris(phenylmethoxy)-cyclohexanone as white cottony crystals (yield: 72.4%).

FT-IR (ν, cm⁻¹, KBr): 3494, 1723, 1497, 1453, 1093, 741, 697

¹H-NMR (300 MHz, CDCl₃, δ): 2.44 (dd, J=3.9, 15.0 Hz), 2.38-2.58 (m), 2.68 (dd, J=3.9, 15.0 Hz), 2.72-2.81 (m), 3.62-3.75 (m), 3.75-3.84 (m), 3.98-4.08 (m), 4.14-4.21 (m), 4.21-4.28 (m), 4.55 (d, J=11.4 Hz), 4.56 (d, J=11.4 Hz), 4.67-5.07 (m), 7.22-7.42 (m)

### PREPARATION EXAMPLE 2

### Preparation of Mixture of 3-Deoxy-2-C-ethenyl-1,5,6-tris-O-(phenylmethyl)-D-epi-inositol and 3-Deoxy-2-C-ethenyl-1.5.6-tris-O-(phenylmethyl)-D-myo-inositol

A mixture (4.33 g, 10 mmol) of [2S-(2α,3β,4α,5α)]-5-hydroxy-2,3,4-tris(phenylmethoxy)-cyclohexanone and [2S-(2α,3β,4α,5β)]-5-hydroxy-2,3,4-tris(phenylmethoxy)-cyclohexanone was dissolved in 90 mL of dry toluene at room temperature under argon. This solution was cooled to −78° C., and thereafter 50 mL of a tetrahydrofuran solution of 1.0 M vinyl magnesium bromide was instilled thereto. The mixture was stirred for 2 hours under the same conditions, and further stirred at room temperature for one hour.

After the termination of the reaction, 100 mL of a 1 mol/L aqueous hydrochloric acid was added slowly to the resulting reaction mixture, and the mixture was then extracted with ethyl acetate (100 mL, once). Its organic phase was washed with water (100 mL, once) and an aqueous saturated sodium chloride (100 mL, once) sequentially. The mixture was dried over anhydrous sodium sulfate. The desiccant was removed by filtration, and the solvent was evaporated at reduced pressure. The residue was dissolved in a mobile phase solvent, and purified with a silica gel column (hexane:ethyl acetate=65:35) to collect the fraction containing the desired product, and the solvent was evaporated at reduced pressure.

The resulting pale yellowish viscous product was crystallized from n-hexane to give 3.03 g of a mixture of 3-deoxy-2-C-ethenyl-1,5,6-tris-O-(phenylmethyl)-D-epi-inositol and 3-deoxy-2-C-ethenyl-1,5,6-tris-O-(phenylmethyl)-D-myo-inositol as a white solid (yield: 65.8%).

FT-IR (ν, cm⁻¹, KBr): 3541, 3472, 3031, 2913, 1497, 1453, 1066, 736, 698

¹H-NMR (300 MHz, CDCl₃, δ): 1.56 (dd, J=3.3, 15.3 Hz), 2.07 (dd, J=3.3, 15.3 Hz), 3.38 (d, J=9.9 Hz), 3.46 (dd, J=3.3, 9.9 Hz), 4.10 (t, J=9.9 Hz), 4.10-4.18 (m), 4.63 (d, J=10.5 Hz), 4.70-4.90 (m), 4.99 (d, J=10.5 Hz), 5.20 (dd, J=1.5, 10.8 Hz), 5.42 (dd, J=1.5, 17.1 Hz), 5.77 (dd, J=10.8, 17.1 Hz), 7.22-7.41 (m)

### PREPARATION EXAMPLE 3

### Preparation of [2R-(2α,3β,4α,5α)]-5-Ethenyl-5-hydroxy-2,3,4-tris(phenylmethoxy)-cyclohexanone

2.99 g of a mixture of 3-deoxy-2-C-ethenyl-1,5,6-tris-O-(phenylmethyl)-D-epi-inositol and 3-deoxy-2-C-ethenyl-1,5,6-tris-O-(phenylmethyl)-D-myo-inositol was dissolved in 15 mL of a dry dimethyl sulfoxide solution and 5.43 mL of triethylamine. 15 mL of a dry dimethyl sulfoxide solution containing 3.10 g of sulfur trioxide pyridine complex was instilled into the mixture, at room temperature. Thereafter, the mixture was stirred for one hour under the same conditions.

After the termination of the reaction, 100 mL of water was added to the resulting reaction mixture, and the mixture was extracted with ethyl acetate (100 mL, once). Its organic phase was washed with a 1 mol/L aqueous hydrochloric acid (100 mL, once) and water (100 mL, once). The mixture was dried over anhydrous sodium sulfate. The desiccant was removed by filtration, and the solvent was evaporated at reduced pressure. The residue was dissolved in a mobile phase solvent, and purified with a silica gel column (hexane:ethyl acetate=7:3). The resulting viscous product was crystallized from n-hexane, to give 2.49 g of [2R-(2α,3β,4α,5α)]-5-ethenyl-5-hydroxy-2,3,4-tris(phenylmethoxy)-cyclohexanone as a white solid (yield: 83.7%).

FT-IR (ν, cm⁻¹, KBr): 3549, 3032, 2908, 2872, 1737, 1497, 1454, 1129, 1027, 755, 700

¹H-NMR (300 MHz, CDCl₃, δ): 2.47 (d, 1H, J=14.7 Hz), 2.55 (d, 1H, J=14.7 Hz), 3.78 (d, 1H, J=9.6 Hz), 4.02 (t, 1H, J=9.6 Hz), 4.15 (d, 1H, J=9.6 Hz), 4.57 (d, 1H, J=11.7 Hz), 4.67 (d, 1H, J=10.5 Hz), 4.76 (d, 1H, J=10.5 Hz), 4.85 (d, 1H, J=10.5 Hz), 4.97 (d, 2H, J=11.7 Hz), 5.25 (dd, 1H, J=1.2, 10.5 Hz), 5.41 (dd, 1H, J=1.2, 16.8 Hz), 5.90 (dd, 1H, J=10.5, 16.8 Hz), 7.20-7.42 (m, 15H)

### EXAMPLE 1

### Preparation of 3,4-Dideoxy-2-C-ethenyl-4-[[2-hydroxy-1-(hydroxymethyl)ethyl]amino]-1,5,6-tris-O-(phenylmethyl)-D-epi-inositol

1.50 g of [2R-(2α,3β,4α,5α)]-5-ethenyl-5-hydroxy-2,3,4-tris(phenylmethoxy)-cyclohexanone and 894 mg of 2-amino-1,3-propanediol were stirred in 25 mL of dry methanol at room temperature for 20 hours. To the resulting mixture, 742 mg sodium borohydride was added under ice-cooling. The mixture was stirred at room temperature for 16 hours.

The solvent was evaporated at reduced pressure. Fifty milliliters of water was added to the residue, and the mixture was extracted with ethyl acetate (50 mL, once). Its organic phase was washed with an aqueous saturated sodium chloride (50 mL, once). The mixture was dried over anhydrous sodium sulfate. The desiccant was removed by filtration, and the solvent was evaporated at reduced pressure. The residue was dissolved in ethyl acetate, and purified with a silica gel column (ethyl acetate).

Thus, 1.27 g of 3,4-dideoxy-2-C-ethenyl-4-[[2-hydroxy-1-(hydroxymethyl)ethyl]amino]-1,5,6-tris-O-(phenylmethyl)-D-epi-inositol was obtained as a white solid (yield: 72.6%).

Melting point: 138°-139° C.

FT-IR(ν, cm⁻¹, KBr): 3580, 3416, 3312, 2879, 1498, 1454, 1362, 1093, 1040, 925, 741, 700

¹H-NMR(300 MHz, CDCl₃, δ): 1.36 (dd, 1H, J=3.0, 15.3 Hz), 1.93 (dd, 1H, J=3.0, 15.3 Hz), 2.74-2.84 (m, 1H), 3.30 (d, 1H, J=9.9 Hz), 3.36-3.45 (m, 1H), 3.61 (dd, 1H, J=4.5, 9.9 Hz), 3.36-3.45 (m, 4H), 4.08 (t, 1H, J=9.9 Hz), 4.60-4.83 (m, 6H), 5.13 (dd, 1H, J=1.8, 10.8 Hz), 5.39 (dd, 1H, J=1.8, 17.4 Hz), 5.76 (dd, 1H, J=10.8, 17.4 Hz), 7.20-7.42 (m, 15H)

### EXAMPLE 2

### Preparation of 3,4-Dideoxy-4-[[2-hydroxy-1-(hydroxymethyl)ethyl]amino]-2-C-(hydroxymethyl)-1,5,6-tris-O-(phenylmethyl)-D-epi-inositol

700 mg of 3,4-dideoxy-2-C-ethenyl-4-[2-hydroxy-1-(hydroxymethyl)ethyl]amino]-1,5,6-tris-O-(phenylmethyl)-D-epi-inositol was dissolved in 30 mL of methylene chloride:methanol (4:1), and the solution was bubbled with ozone (O₃) at −78° C. (time period required for the raw material spots to disappear on TLC: about 4 hours).

198 mg of sodium borohydride was added to the resulting reaction mixture, and the mixture was reacted at room temperature for 1 hour. Thereafter, 20 mL of a 1 mol/L aqueous hydrochloric acid was added thereto, and the pH was adjusted to 4.

Next, the pH was adjusted to 12 with a 2 mol/L aqueous sodium hydroxide, and the mixture was then extracted with chloroform (50 mL, once). Its organic phase was washed with water (50 mL, once) and then with a saturated solution of sodium chloride (50 mL, once). Thereafter, the mixture was dried over anhydrous sodium sulfate. The desiccant was removed by filtration, and the solvent was evaporated at reduced pressure. The residue was dissolved in a mobile phase solvent and purified with a silica gel column (chloroform:methanol=9:1), to give 604 mg of 3,4-dideoxy-4-[[2-hydroxy-1-(hydroxymethyl)ethyl]amino]-2-C-(hydroxymethyl)-1,5,6-tris-O-(phenylmethyl)-D-epi-inositol as a white foamy solid (yield: 85.5%).

FT-IR (ν, cm⁻¹, KBr): 3401, 2874, 1497, 1454, 1359, 1069, 734, 697

¹H-NMR (300 MHz, CDCl₃, δ): 1.26 (dd, 1H, J=3.0, 15.3 Hz), 2.10 (dd, 1H, J=3.0, 15.3 Hz), 2.76-2.88 (m, 1H), 3.17-3.26 (m, 1H), 3.33 (d, 1H, J=9.9 Hz), 3.40-3.56 (m, 4H), 3.62 (dd, 1H, J=4.5, 9.9 Hz), 3.66-3.85 (m, 4H), 4.16 (t, 1H, J=9.9 Hz), 4.60-5.00 (m, 6H), 7.22-7.40 (m, 15H)

### EXAMPLE 3

### Preparation of 3,4-Dideoxy-4-[[2-hydroxy-1-(hydroxymethyl)ethyl]amino]-2-C-(hydroxymethyl)-D-epi-inositol

100 mg of 3,4-dideoxy-4-[[2-hydroxy-1-(hydroxymethyl)ethyl]amino]-2-C-(hydroxymethyl)-1,5,6-tris-O-(phenylmethyl)-D-epi-inositol was dissolved in 1.9 mL of methanol at room temperature under argon. Thereafter, 0.1 mL of 90% formic acid and 20 mg of palladium black were added thereto, and the mixture was stirred at 60° C. for 6 hours. The catalyst was removed from the reaction mixture by filtration, and the filtrate was washed with 3 mL of a mixed solvent of methanol and water [methanol:water=1:1]. Thereafter, the solvent was evaporated at reduced pressure from the filtrate.

The residue was subjected to a column chromatography of a strongly acidic cation exchanging resin (commercially available from Dow Chemical under the trade name of DOWEX 50W×8) (H⁺-type). After the column was washed with water, the desired product was eluted with 0.5 N aqueous ammonia. The eluted fraction was evaporated at reduced pressure, and the residue was subjected to a column chromatography of a strongly basic anion exchanging resin (commercially available from ORGANO Corporation under the trade name of AMBERLITE CG-50) (NH₄-type), and eluted with water. The eluted fraction was evaporated at reduced pressure, and the residue was refluxed with 5 μL of ethanol. Thereafter, the reaction mixture was allowed to stand overnight in a thermostatic chamber at 5° C.

The solid precipitate was collected by filtration, to give 33 mg of 3,4-dideoxy-4-[[2-hydroxy-1-(hydroxymethyl)ethyl]amino]-2-C-(hydroxymethyl)-D-epi-inositol] (yield: 65.0%).

FT-IR(ν, cm⁻¹, KBr): 3459, 3297, 2955, 1089, 1057, 1037, 569

¹H-NMR (300 MHz, D₂O, δ): 1.51 (dd, 1H), 2.05 (dd, 1H), 2.83-2.90 (m, 1H), 3.35-3.85 (m, 10H)

The scheme showing the summary of the preparation process of the present invention as explained above is as follows.

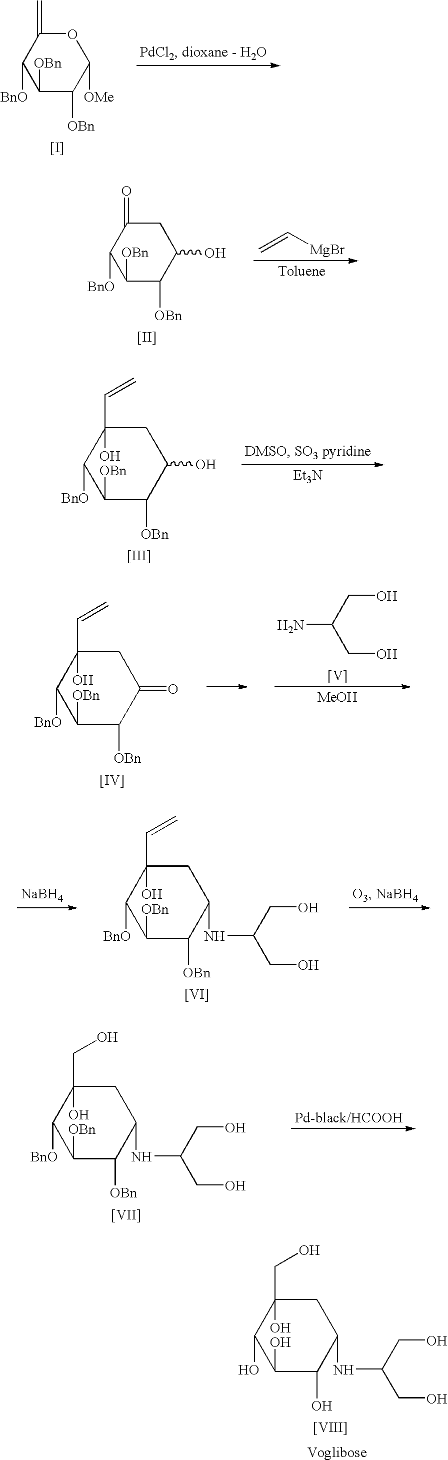

### INDUSTRIAL APPLICABILITY

According to the present invention, voglibose can be conveniently prepared in a low cost with a safe process. Also, according to the present invention, an inositol derivative represented by the formula (VI), which is an intermediate for voglibose, which can be suitably used in the preparation of voglibose.

1. An inositol derivative represented by the formula (VI): 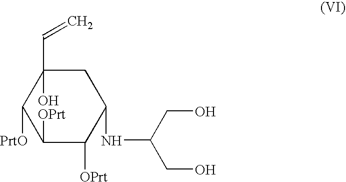
wherein Prt is a protecting group of hydroxyl group.

2. The inositol derivative according to claim 1, wherein Prt is a benzyl group, an acyl group or a silyl group, each of which may have a substituent.

3. A process for preparing an inositol derivative represented by the formula (VI): 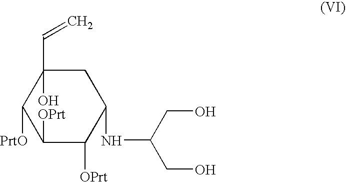
wherein Prt is a protecting group of hydroxyl group,
characterized in that a cyclohexanone compound represented by the formula (IV): 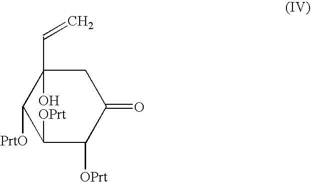
wherein Prt is as defined above,
is dihydroxyaminated using a dihydroxyaminating agent and a reducing agent.

4. The process for preparing an inositol derivative according to claim 3, wherein the dihydroxyaminating agent is 2-amino-1,3-propanediol represented by the formula (V):

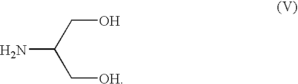

5. The process for preparing an inositol derivative according to claim 3 or 4, wherein Prt is a benzyl group, an acyl group or a silyl group, each of which may have a substituent.

6. A process for preparing voglibose represented by the formula (VIII): 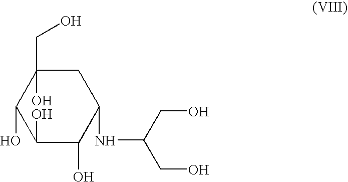
characterized in that an inositol derivative represented by the formula (VI): 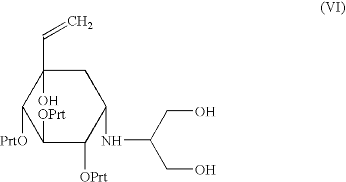
wherein Prt is a protecting group of hydroxyl group,
is oxidized to give an inositol compound represented by the formula (VII): 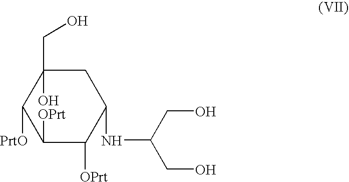
wherein Prt is as defined above, and
the protecting group, Prt of the inositol compound is deprotected.

7. The process for preparing voglibose according to claim 6, wherein Prt is a benzyl group, an acyl group or a silyl group, each of which may have a substituent.
